(19) United States
(12) Patent Application Publication
(10) Pub. No.: US 2012/0121558 A1
(43) Pub. Date: May 17, 2012

(54) COMPOSITIONS AND METHODS FOR TREATING PLASMA PROTEIN DEFICIENCY DISORDERS

(76) Inventors: Keith Leonard March, Carmel, IN (US); Elliot David Rosen, South Bend, IN (US); Katherine Marcucci, Philadelphia, PA (US); Katherine High, Philadelphia, PA (US)

(21) Appl. No.: 13/109,850

(22) Filed: May 17, 2011

Related U.S. Application Data
(60) Provisional application No. 61/345,423, filed on May 17, 2010.

(51) Int. Cl. A61K 35/12; C12N 5/10; A61P 7/04; C12N 5/071

(52) U.S. Cl. 424/93.21; 424/93.7; 435/325

(57) ABSTRACT

The disclosure of the present application provides compositions and methods for treating a blood disorder. In at least one embodiment of a method for treating a patient with a plasma protein deficiency disorder, the method comprises the steps of administering a cell-based composition to a patient with a plasma protein deficiency disorder to treat the plasma protein deficiency disorder, where the cell-based composition comprises a mammalian adipose stromal cell that is capable of effectuating the production of a plasma protein within the patient.

### PRIORITY

The present United States utility patent application is related to, and claims the priority benefit of, U.S. Provisional Patent Application Ser. No. 61/345,423, filed May 17, 2010, the contents of which are hereby incorporated by reference in their entirety into this disclosure.

### BACKGROUND

The discovery of pluripotent cells in adipose tissue has revealed a novel source of cells that may be used for autologous cell therapy to ameliorate deficiencies in patients. These pluripotent cells reside in the “stromal” or “non-adipocyte” fraction of the adipose tissue, and were previously considered to be pre-adipocytes (i.e. adipocyte progenitor cells), however recent data suggests a much wider differentiation potential. Zuk et al. were able to establish differentiation of such subcutaneous human adipose stromal cells (“ASCs”) in vitro into adipocytes, chondrocytes and myocytes. (Zuk P A, et al. Mol Biol Cell 13(12):4279-4295, 2002.) These findings were extended in a study by Erickson et al., which showed that human ASCs could differentiate in vivo into chondrocytes following transplantation into immune-deficient mice. Erickson G R, el al. Biochem Biophys Res Commun. 2002; 290:763-669. More recently, it was demonstrated that human ASCs were able to differentiate into neuronal cells, osteoblasts cardiomyocyte, and endothelial cells.

Hemophilia is a group of hereditary genetic disorders that impair the body's ability to control blood clotting or coagulation, which is used to stop bleeding when a blood vessel is damaged. Two major classes of hemophilia include hemophilia A, which has a deficiency in clotting factor VIII (FVIII), and hemophilia B, which has a deficiency in clotting factor IX (FIX). Because of these deficiencies, when a blood vessel is injured, a temporary scab does form at the site of injury, but the missing coagulation factors prevent fibrin formation, which is necessary to maintain the blood clot.

Given that hemophilia presents a life altering condition, and that current avenues of treatment are inadequate, there is a need for improved methods of therapy or prevention of hemophilia. Further, there is also a need for improved treatments for other blood clotting disorders and plasma protein deficiency disorders in general.

### BRIEF DESCRIPTION

Disclosed herein are various methods and compositions for treating a patient having a plasma protein deficiency disorder. At least some of the methods and compositions disclosed involve the use of mammalian adipose stromal cells.

In at least one embodiment of a method for treating a patient with a plasma protein deficiency disorder, the method comprises the steps of administering a cell-based composition to a patient with a plasma protein deficiency disorder to treat the plasma protein deficiency disorder, the cell-based composition comprising a mammalian adipose stromal cell capable of effectuating the production of a plasma protein within the patient. Optionally, the plasma protein is selected from the group consisting of Factor VIII, B-domainless Factor VIII, Factor VII, Factor IX, Factor X, protein C, and prothrombin. Further, the method for treating a patient with a plasma protein deficiency disorder may also comprise a step of introducing an isolated nucleotide sequence encoding the plasma protein into the mammalian adipose stromal cell, where the plasma protein selected from the group consisting of Factor VIII, B-domainless Factor VIII, Factor VII, Factor IX, Factor X, protein C, and prothrombin.

In at least one embodiment of a method for treating a patient with a plasma protein deficiency disorder, the step of introducing the isolated nucleotide sequence is performed at a multiplicity of infection selected from the group consisting of about 1.0 e5 to about 1.0 e7, about 5.0 e5 to about 5.0 e6, and about 5.0 e5 to about 1.0 e6. The mammalian adipose stromal cell of the cell-based composition administered to the patient may also have been previously isolated from the patient. Further, the step of administering the mammalian adipose stromal cell of an embodiment of the method may be performed by a route selected from a group consisting of intravenous injection, intramuscular injection, subcutaneous injection, retrograde venous injection, arterial injection, surgical implantation, and intraocular placement. Moreover, the plasma protein deficiency disorder may be selected from a group consisting of hemophilia type A and hemophilia type B.

In at least one embodiment of the method for treating a patient with a plasma protein deficiency disorder, the mammalian adipose stromal cell is selected from the group consisting of a CD10+ mammalian adipose stromal cell, a CD13+ mammalian adipose stromal cell, a CD34+ mammalian adipose stromal cell, a CD34− mammalian adipose stromal cell, a CD45+ mammalian adipose stromal cell, a CD45− mammalian adipose stromal cell, a CD90+ mammalian adipose stromal cell, a CD90− mammalian adipose stromal cell, a CD140a+ mammalian adipose stromal cell, a CD140a− mammalian adipose stromal cell, a CD140b+ mammalian adipose stromal cell, and a CD140b− mammalian adipose stromal cell. Further, the method may also comprise the step of differentiating the mammalian adipose stromal cell to increase the expression of at least one hepatocyte characteristic, where the hepatocyte characteristic may be selected from the group consisting of alpha-fetoprotein, cytochrome P450 family 3 subfamily A (CYP3A), albumin, and hepatocyte nuclear factor alpha.

In at least one embodiment of the method for treating a patient with a plasma protein deficiency disorder, the method comprises the step of administering a cell-based composition to a patient with a plasma protein deficiency disorder to treat the plasma protein deficiency disorder, the cell-based composition comprising a mammalian adipose stromal cell capable of effectuating the promotion of a plasma protein within the patient, wherein the mammalian adipose stromal cell comprises an isolated nucleotide sequence encoding a protein capable of compensating for the plasma protein deficiency disorder in the patient. The mammalian adipose stromal cell may also be differentiated to express at least one hepatocyte characteristic, such as Factor VIII, B-domainless Factor VIII, Factor VII, Factor IX, Factor X, protein C, and prothrombin. Additionally, the mammalian adipose stromal cell may be originally isolated from the patient that the differentiated adipose stromal cell is administered. Further, the step of administering the mammalian adipose stromal cell may be performed by a route selected from a group consisting of intravenous injection, intramuscular injection, subcutaneous injection, retrograde venous injection, arterial injection, surgical implantation, and intraocular placement.

In at least one embodiment of the method for treating a patient with a plasma protein deficiency disorder, the step of administering a cell-based composition comprises administering a cell-based composition comprising a mammalian adipose stromal cell, and at least one secondary cell selected from the group consisting of a mammalian endothelial cell, a mammalian endothelial progenitor cell, an unmodified adipose stromal cell, or a combination thereof. Further, the at least one secondary cell may be effective to promote localized vascularization in the patient. Additionally, the cell-based composition may be provided in a form selected from the group consisting of a matrix form and a capsule form. Moreover, the form may be effective to prevent degradation of the cell-based composition by an immunogenic cell for a period of time.

In at least one embodiment of the method or composition of the present disclosure, the mammalian adipose stromal cell is selected from the group consisting of a CD10+ mammalian adipose stromal cell, a CD13+ mammalian adipose stromal cell, a CD34+ mammalian adipose stromal cell, a CD34− mammalian adipose stromal cell, a CD45+ mammalian adipose stromal cell, a CD45− mammalian adipose stromal cell, a CD90+ mammalian adipose stromal cell, a CD90− mammalian adipose stromal cell, a CD140a+ mammalian adipose stromal cell, a CD140a− mammalian adipose stromal cell, a CD140b+ mammalian adipose stromal cell, and a CD140b− mammalian adipose stromal cell.

In at least one embodiment of a composition to treat a plasma protein deficiency disorder, the composition comprises a mammalian adipose stromal cell, wherein the composition is effective to treat the plasma protein deficiency disorder by effectuating the production of a plasma protein within the patient, the plasma protein being selected from the group consisting of Factor VIII, B-domainless Factor VIII, Factor VII, Factor IX, Factor X, protein C, and prothrombin. The mammalian adipose stromal cell may also comprise an isolated nucleotide sequence encoding a protein capable of compensating for the plasma protein deficiency in the patient. For example, the isolated nucleotide sequence may encode a protein selected from the group consisting of Factor VIII, B-domainless Factor VIII, Factor VII, Factor IX, Factor X, protein C, and prothrombin. Additionally, the composition may be provided in a form selected from the group consisting of an intravenous injectable form, a surgically-implantable form, a intramuscular injectable form, a subcutaneous injectable form, a retrograde venous injectable form, an arterial injectable form, and an intraocular placeable form. Further, the composition may further comprise a biologically-compatible carrier.

In at least one embodiment of a composition to treat a plasma protein deficiency disorder, the composition comprises at least one secondary cell selected from the group consisting of a mammalian endothelial cell, a mammalian endothelial progenitor cell, an unmodified adipose stromal cell, or a combination thereof. Further, the at least one secondary cell may be effective to promote localized vascularization in the patient. Additionally, the composition may be provided in a form selected from the group consisting of a matrix form and a capsule form. Optionally, the form may be effective to prevent degradation of the cell-based composition by an immunogenic cell for a period of time.

### BRIEF DESCRIPTION OF THE DRAWINGS

FIG. 1 shows a timeline of human adipose stromal cell (hASC) hepatocyte differentiation and representative micrographs showing the morphology of cell types at each stage of differentiation, according to a prior art disclosure;

FIG. 2 shows a flowchart depicting the steps for treating a patient with a plasma protein deficiency disorder, according to at least one embodiment of the present disclosure;

FIG. 3 shows a timeline of hASC hepatocyte differentiation and details regarding transduction of the differentiated cells, according to at least one embodiment of the present disclosure;

FIG. 4 shows a graphical representation of the activity level of human factor IX (FIX) secreted from hepatocyte-like hASC, according to at least one embodiment of the present disclosure;

FIG. 5 shows a graphical representation of the level of FIX secreted from hepatocyte-like hASC, according to at least one embodiment of the present disclosure; and

FIG. 6 shows a visualization of liver specific markers produced by hASC cells during differentiation as indicated by reverse transcriptase-PCT analysis, according to at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The disclosure of the present application provides various compositions and methods for cell-based therapies. The compositions and methods disclosed herein are useful to treat patients with plasma protein deficiencies through the introduction of mammalian adipose stromal cells (ASCs).

ASCs are isolated from human, and other mammalian, subcutaneous adipose tissue according to the method of Zuk et al. ASCs are predominantly localized in the peri-endothelial layer of the vessels in vivo (in adipose tissue), and are phenotypically and functionally equivalent to pericytes associated with microvessels. The ASCs may, in at least one illustrative example, be isolated at a level of about 10⁸ cells per 100 ml of lipoaspirate. Further, following isolation, the isolated ASCs may be cultured on tissue culture plastic in EGM-2mv medium. In this medium, ASCs can expand to about 1000-fold over a 10 day period. Moreover, ASCs isolated from humans (hASCs) routinely secrete a wide variety of bioactive molecules, such as VEGF, HGF, and GM-CSF, which participate in stimulation of EC survival and proliferation and stabilization of endothelial networks formed on the surface of Matrigel. Following the isolation of hASC, these cells may be differentiated toward hepatocyes, such as, for example, by the method of Talens-Visconti, R. et al. (World J. Gastroenterol. 12: 5834-45 (2006), the contents of which are incorporated herein in their entirety). Differentiation of hASC may be performed using appropriate growth factors, such as those included in Talens-Visconti, et al.

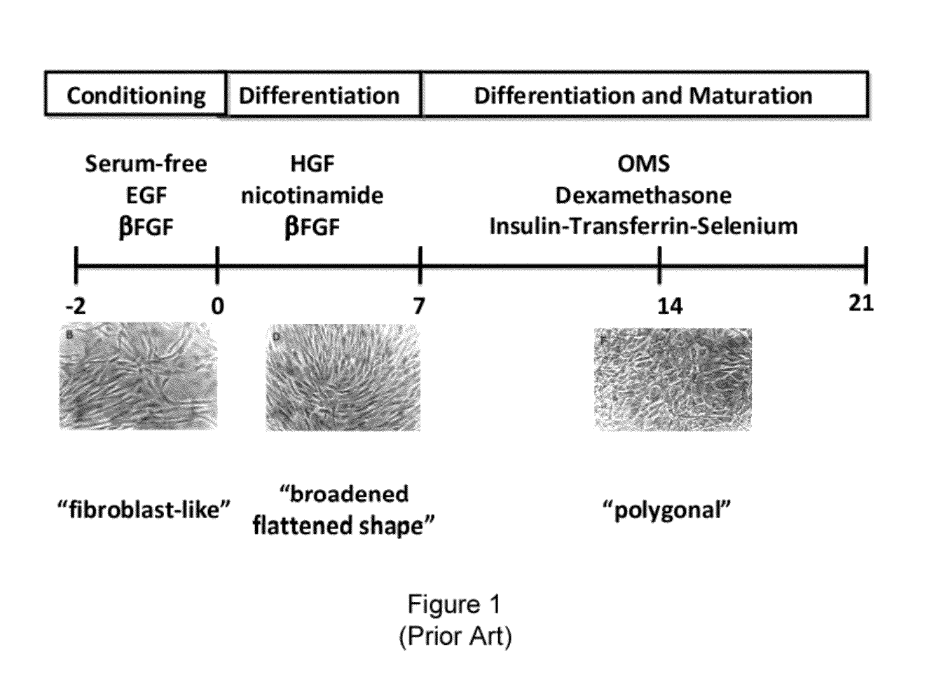

Turning to FIG. 1, a timeline depicting the prior art method of Talens-Visconti, R. et al. for the differentiation of hASC is shown. Specifically, the timeline shows a three step process, where the cells are first conditioned, then differentiated, and finally differentiated and matured. The Conditioning step of hASC occurs through the use of serum-free EGF and βFGF between day −2 and day 0, where the hASC have a fibroblast-like morphology. Following the Conditioning step is the Differentiation step between day 0 and day 7, where the hASC are exposed to HGF, nicotinamined, and βFGF, and undertake a “broadened flattened shape.” Finally, between day 7 and day 21, the hASC are in the Differentiation and Maturation stage, where the hASC are exposed to OMS, Dexamethasone, and Insulin-Transferrin-Selenium, and take on a polygonal morphology.

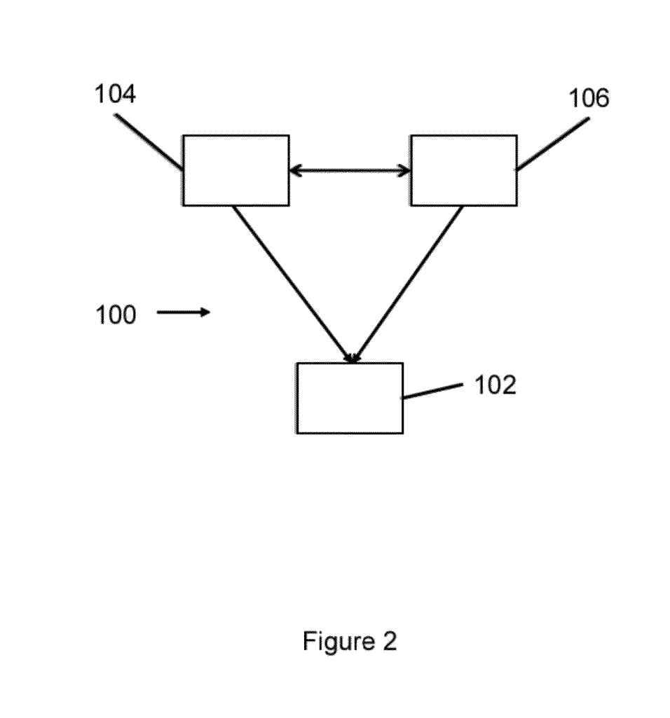

Referring to FIG. 2, at least one embodiment of a method 100 of treating a patient with a plasma protein deficiency disorder is depicted. Exemplary method 100 comprises the step of administering a cell-based composition to a patient with a plasma protein deficiency disorder to treat the plasma protein deficiency disorder, where the cell-based composition comprises a mammalian adipose stromal cell capable of effectuating the production of a plasma protein within the patient (exemplary administration step 102). Additionally, exemplary method 100 may further comprise the step of introducing an isolated nucleotide sequence encoding the plasma protein into the mammalian adipose stromal cell, where the plasma protein is selected from the group consisting of Factor VIII, B-domainless Factor VIII, Factor VII, Factor IX, Factor X, protein C, and prothrombin (exemplary introduction step 104). Moreover, exemplary method 100 may also comprise the step of differentiating the mammalian adipose stromal cell to increase the expression of at least one hepatocyte characteristic (exemplary differentiation step 106). Specifically, the at least one hepatocyte characteristic may include one or more of alpha-fetoprotein, cytochrome P450 family 3 subfamily A (CYP3A), albumin, and hepatocyte nuclear factor 4-alpha. In at least one embodiment, introduction step 104 and/or differentiation step 106 may both occur prior to administration step 102, and differentiation step 106 may occur through the method of Talens-Visconti, R. et al., as depicted in FIG. 1.

The step of administering 102 the adipose stromal cell into a patient, according to at least one exemplary embodiment, may be performed by a route selected from a group consisting of intravenous injection, intramuscular injection, subcutaneous injection, retrograde venous injection, arterial injection, surgical implantation, and intraocular placement. Optionally, the administering 102 a cell-based composition comprises administering a cell-based composition comprising a mammalian adipose stromal cell, and at least one secondary cell selected from the group consisting of a mammalian endothelial cell, a mammalian endothelial progenitor cell, an unmodified adipose stromal cell, or a combination thereof. Further, the at least one secondary cell may be effective to promote localized vascularization in the patient.

Additionally, the cell-based composition may be provided in a form selected from the group consisting of a matrix form and a capsule form. The form, in at least one example, may comprise collagen, fibronectin, a combination thereof, or any acceptable and biocompatible form. Moreover, the form may be effective to prevent degradation of the cell-based composition by an immunogenic cell for a period of time. Such a period of time may in an exemplary embodiment be adequate for implantation of the cell-based composition at the site of therapy in the patient. Specifically, an exemplary period of time may be selected from the group consisting of 1 minute or less, 5 minutes or less, 10 minutes or less, 15 minutes or less, and 1 hour or less.

Introduction 104 of the isolated nucleotide sequence into the adipose stromal cell may be conducted through any number of appropriate and effective means to introduce DNA or RNA into a mammalian cell. For example, the nucleotide sequence may be first inserted onto an adeno-associated virus (AAV), such as AAV2-CMV, and then introduced into the adipose stromal cell. Additionally, other genetic vectors may be used, including viral vectors and artificial chromosomal constructs incorporating sequences that encode the desired proteins. Multiplicity of infection ranges for the introduction of the AAV into ASC in an exemplary introduction step 104 may be about 1.0 e5 to about 1.0 e7, about 5.0 e5 to about 5.0 e6, and about 5.0 e5 to about 1.0 e6.

Exemplary differentiation step 106 may occur through any means sufficient to alter the protein expression pattern of a mammalian ASC to that more similar to a hepatocyte. In such a differentiation, the differentiated ASC expresses at least one hepatocyte characteristic. Exemplary hepatocyte characteristics that may be expressed by the differentiated ASC include, but are not limited to, alpha-fetoprotein, cytochrome P450 family 3 subfamily A (CYP3A), albumin, and hepatocyte nuclear factor alpha. One such method for differentiation of ASC is through the method described by Talens-Visconti, et al.

Alternately, the adipose stromal cell to be administered to the patient may be undifferentiated, but transduced to include an isolated nucleotide sequence encoding a protein capable of compensating for the plasma protein deficiency. Moreover, the adipose stromal cell to be administered to the patient may be a mixture of differentiated adipose stromal cells and undifferentiated adipose stromal cells. Further, the differentiated adipose stromal cell may also be transduced in a like manner to compensate for the plasma protein deficiency.

In an exemplary embodiment of the cell-based therapy method of the present disclosure, the nucleotide sequence may encode a protein selected from the group consisting of Factor VIII, Factor IX, Factor X, protein C, and prothrombin. Further, the step of administering the adipose stromal cell may be performed by a route selected from a group consisting of intravenous injection, intramuscular injection, subcutaneous injection, retrograde venous injection, arterial injection, surgical implantation, and intraocular placement. Moreover, the adipose stromal cell used in the cell-based therapy method may be originally isolated from the patient that the differentiated adipose stromal cell is administered.

Given that either or both of introduction step 104 and differentiation step 106 may occur prior to administering step 102, the mammalian ASC may or may not contain isolated DNA or have undergone the process of differentiation prior to administration into a patient having a plasma protein deficiency. Accordingly, the administered cell-based composition may comprise one or more of (a) undifferentiated ASC introduced with isolated DNA, (b) undifferentiated ASC not containing isolated DNA, (c) differentiated ASC introduced with isolated DNA, and (d) differentiated ASC not containing isolated DNA. Further, the cell-based composition may, in some embodiments, further comprise a biological carrier, such as matrigel.

Embodiments of a cell-based composition, as described herein, may also include co-implantation of cells capable of forming neo-tissues or vasculature. For example, undifferentiated (or differentiated) adipose stromal cells may be introduced with endothelial cells to provide vascular support as well as drainage for a secreted factor.

In another exemplary embodiment of a cell-based therapy method of the present disclosure, the step of administering the differentiated adipose stromal cell compensates for the plasma protein deficiency in the patient. The plasma protein deficiency in an exemplary embodiment may be a clotting disorder, such as hemophilia type A, hemophilia type B, Factor V Leiden, protein C deficiency, protein S deficiency, anti-thrombin deficiency, or a prothrombin 20210A mutation.

According to at least one embodiment of a composition to treat a plasma protein deficiency disorder of the present disclosure, the composition comprises an embodiment of a mammalian ASC, where the composition is effective to treat the plasma protein deficiency disorder. The treatment, in at least one embodiment, may occur by effectuating the production of a plasma protein within the patient. Exemplary plasma proteins, which may be produced, include Factor VIII, B-domainless Factor VIII, Factor VII, Factor IX, Factor X, protein C, and prothrombin.

In an exemplary embodiment of the composition of the present disclosure, the composition may be provided in a form selected from the group consisting of an intravenous injectable form, a surgically-implantable form, a intramuscular injectable form, a subcutaneous injectable form, a retrograde venous injectable form, an arterial injectable form, and an intraocular placeable form. Optionally, the composition may also comprise a biologically-compatible carrier.

Further, an exemplary composition may be provided in a form selected from the group consisting of a matrix form and a capsule form. The form, in at least one example, may comprise collagen, fibronectin, a combination thereof, or any acceptable and biocompatible form. Moreover, the form may be effective to prevent degradation of the cell-based composition by an immunogenic cell for a period of time. Such a period of time may in an exemplary embodiment be adequate for implantation of the cell-based composition at the site of therapy in the patient. An exemplary period of time in an embodiment of a composition or method of the present disclosure may be selected from the group consisting of 1 minute or less, 5 minutes or less, 10 minutes or less, 15 minutes or less, and 1 hour or less.

### EXAMPLES

### Example 1

A majority of human ASCs (hASCs) isolated as described Zuk et al. and additionally enriched by attachment to tissue culture plastic, express the stem cell marker CD34 (in the first days of culture), as well as co-express several mesenchymal cell markers (CD10+/CD13+/CD90+) and pericyte markers (CD140a+/CD140b+/NG2+).

### Example 2

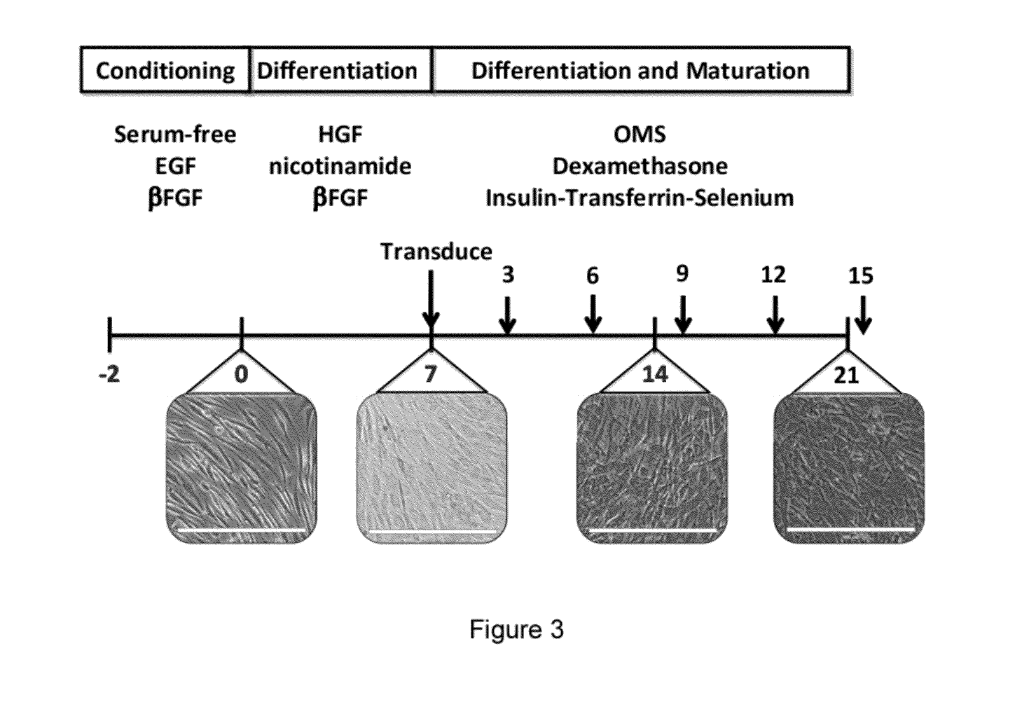

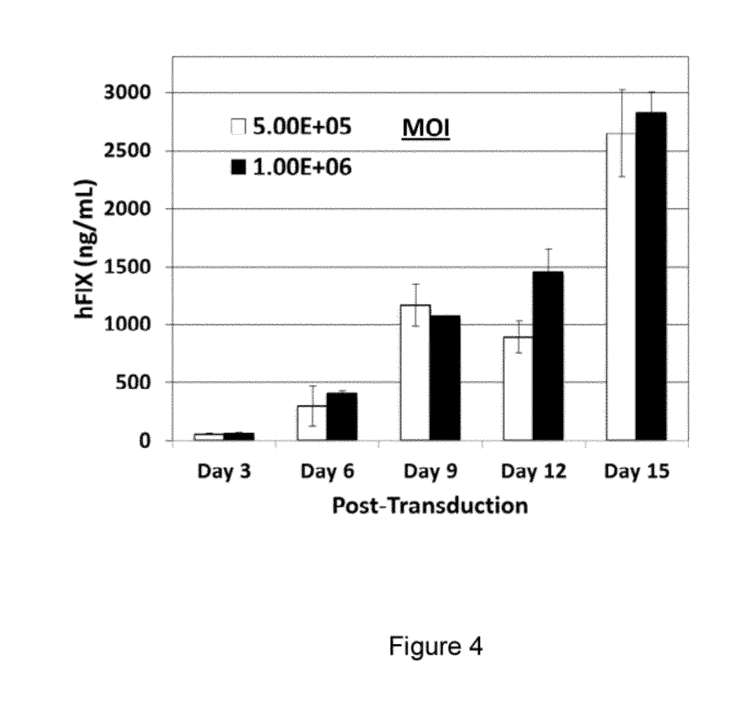

To modify the level of hFIX, the hepatocyte-like hASCs were transduced at day 7 with an AAV-2 vector delivering hFIX under control of the CMV promoter at two MOIs, 5e5 and 1e6 vg/cell (See FIG. 3). Following transduction, supernatant hFIX levels were measured by Enzyme-linked immunosorbent assay (ELISA) every 3 days for 15 days post-transduction (See FIG. 4). The level of hFIX in the supernatant of untransduced differentiated cells was below the lower limit of detection (20 ng/mL) of the ELISA. However, the level of hFIX from transduced cells was detected in the supernatant as early as 3 days post-transduction, and peak levels at day 15 post-transduction were 3.5 and 3.7 μg/10⁶ cells/24 hours for MOIs 5e5 and 1e6 vg/mL, respectively. These levels are higher than those found in the literature for hFIX secretion from a primary or stem cell population transduced by a viral vector.

### Example 3

The clotting activity of the secreted hFIX was also measured by activated partial thromboplastin time (aPTT) on days 9 and 15 post-transduction. At a MOI of 5e5 the specific activity was 173 U/mg at day 9 and 215 U/mg at day 15. For the MOI of 1e6 the specific activity was 226 U/mg at day 9 and 252 U/mg at day 15. Wild-type hFIX specific activity was observed on both days for both MOIs. Therefore, these studies indicate that hASC secrete fully functional hFIX and may be considered as a source for an autologous cell-based treatment following ex vivo gene therapy for the treatment of hemophilia.

Further, expression of hFIX from hepatocyte differentiated hASC was shown to be higher than that expressed by porcine bone marrow stem cells or human primary myoblasts (Table 1). Both porcine bone marrow stem cells and human primary myoblasts were transfected with retrovirus containing hFIX (See Krebsbach P H, et al. (2003) Journal of Gene Medicine. 5: 11-17 (Porcine Bone Marrow Stem cell analysis); and Wen J, et al. (2007) Journal of Gene Medicine. 9: 1002-1010 (Human Primary Myoblast analysis). In each of these analysis, the hepatocyte differentiated human ASC transfected with AAV-hFIX expressed more hFIX than porcine bone marrow stem cells and human primary myoblasts (3529-3775 ng/10⁶ cells/24 hours compared to 360 or 1289 ng/10⁶ cells/24 hours).

TABLE 1
hFIX Expression Between Transfected Cell Types

| | | In vitro Peak hFIX |
|---|---|---|
| | | levels (ng/10⁶ |
| Cell Type | Vector Type | cells/24 hours) |
| Porcine Bone Marrow | Retro viral | 360 |
| Stem cells | | |
| Human Primary | Retro viral | 1289 |
| Myoblasts | | |
| Hepatocyte differentiated | AAV | 5e5: 3529 |
| Human Adipose Stromal | | 1e6: 3775 |
| Cells | | |

### Example 4

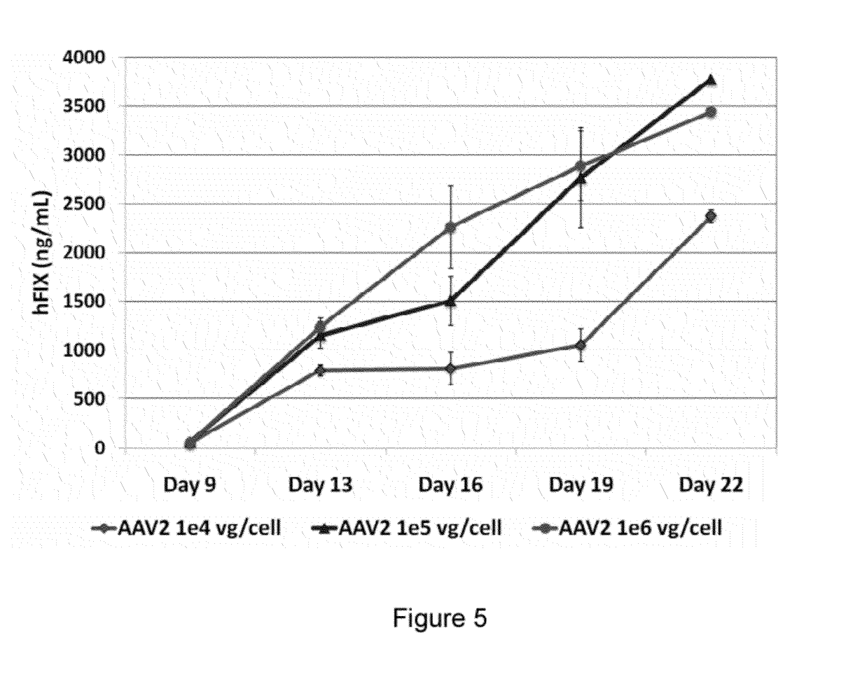

To determine the dose response of production of hFIX based on level of MOI of AAV-CMV-hFIX transducted, a time course analysis was conducted from day 9 to day 22 following the start of differentiation (See FIG. 5). In this analysis, MOI levels of 1e4 vg/cell, 1e5 vg/cell, and 1e6 vg/cell were examined. From this analysis, it was determined that 1e4 vg/cell produces a level of hFIX of 2.7 μg/10⁶ cells/24 hour; 1e5 vg/cell produces a level of hFIX of 4.3 μg/10⁶ cells/24 hour; and 1e6 vg/cell produces a level of hFIX of 4.0 μg/10⁶ cells/24 hour.

### Example 5

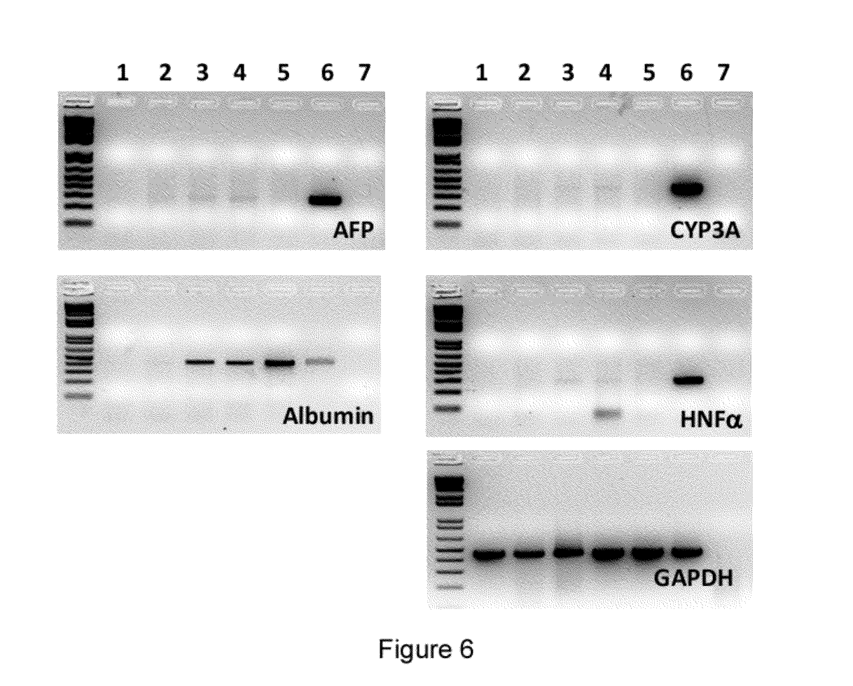

The process of differentiation of ASCs to hepatocyte cells was monitored by Reverse Transcriptase-Polymerase Chain Reaction (RT-PCR) for liver-specific markers, such as α-fetoprotein, albumin, HNF4a and CYP3A. Samples examined by this process include those from: day 0 (lane 1), day 7 (lane 2), day 14 (lane 3), day 21 (lane 4), HHL5 (lane 6), human fetal liver (lane 6), and reverse transcriptase blank (lane 7). By the end of the 21-day protocol, the differentiated hASCs expressed all of the liver-specific markers as shown in FIG. 6. Additionally, the relative expression levels of γ-glutamyl carboxylase, Vitamin K epoxide reductase, quinone reductase and PACE/furin were also determined for these cells by RT-qPCR. These genes are required for the Vitamin K-dependent modifications of hFIX responsible for full clotting activity and are expressed in the hASC population prior to and throughout the differentiation protocol. Relative expression levels of FIX as compared to that of the hepatocyte cell line HHL5 are shown in Table 2.

TABLE 2
Relative FIX Levels

| | HHL5 | hASC D₀ | hASC D₇ | hASC D₁₄ | hASC D₂₁ |
|---|---|---|---|---|---|
| VKOR | 1.0 | 1.6 | 3.9 | 1.6 | 1.8 |
| NQO1 | 1.0 | 1.9 | 2.2 | 1.7 | 0.7 |
| GGCX | 1.0 | 1.0 | 1.1 | 0.5 | 0.6 |
| PACE | 1.0 | 2.1 | 3.8 | 1.7 | 1.5 |

While various embodiments of compositions for treatment of plasma protein deficiency disorders and methods for using the same have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the spirit and scope of the present disclosure.

1. A method for treating a patient with a plasma protein deficiency disorder, the method comprising the step of:
administering a cell-based composition to a patient with a plasma protein deficiency disorder to treat the plasma protein deficiency disorder, the cell-based composition comprising a mammalian adipose stromal cell capable of effectuating the production of a plasma protein within the patient.

2. The method of claim 1, wherein the plasma protein is selected from the group consisting of Factor VIII, B-domainless Factor VIII, Factor VII, Factor IX, Factor X, protein C, and prothrombin.

3. The method of claim 1, further comprising the step of introducing an isolated nucleotide sequence encoding the plasma protein into the mammalian adipose stromal cell, the plasma protein selected from the group consisting of Factor VIII, B-domainless Factor VIII, Factor VII, Factor IX, Factor X, protein C, and prothrombin.

4. The method of claim 3, wherein the step of introducing the isolated nucleotide sequence is performed at a multiplicity of infection selected from the group consisting of about 1.0 e5 to about 1.0 e7, about 5.0 e5 to about 5.0 e6, and about 5.0 e5 to about 1.0 e6.

5. The method of claim 1, wherein the mammalian adipose stromal cell of the cell-based composition administered to the patient was previously isolated from the patient.

6. The method of claim 1, wherein the step of administering the mammalian adipose stromal cell is performed by a route selected from a group consisting of intravenous injection, intramuscular injection, subcutaneous injection, retrograde venous injection, arterial injection, surgical implantation, and intraocular placement.

7. The method of claim 1, wherein the plasma protein deficiency disorder is selected from a group consisting of hemophilia type A, hemophilia type B, Factor V Leiden, protein C deficiency, protein S deficiency, anti-thrombin deficiency, and prothrombin 20210A mutation.

8. The method of claim 1, wherein the mammalian adipose stromal cell is selected from the group consisting of a CD10+ mammalian adipose stromal cell, a CD13+ mammalian adipose stromal cell, a CD34+ mammalian adipose stromal cell, a CD34− mammalian adipose stromal cell, a CD45+ mammalian adipose stromal cell, a CD45− mammalian adipose stromal cell, a CD90+ mammalian adipose stromal cell, a CD90− mammalian adipose stromal cell, a CD140a+ mammalian adipose stromal cell, a CD140a− mammalian adipose stromal cell, a CD140b+ mammalian adipose stromal cell, and a CD140b− mammalian adipose stromal cell.

9. The method of claim 1, further comprising the step of differentiating the mammalian adipose stromal cell to increase the expression of at least one hepatocyte characteristic.

10. The method of claim 9, wherein the at least one hepatocyte characteristic is selected from the group consisting of alpha-fetoprotein, cytochrome P450 family 3 subfamily A (CYP3A), albumin, and hepatocyte nuclear factor alpha.

11. The method of claim 1, wherein the step of administering a cell-based composition comprises administering the cell-based composition comprising a mammalian adipose stromal cell, and at least one secondary cell selected from the group consisting of a mammalian endothelial cell, a mammalian endothelial progenitor cell, an unmodified adipose stromal cell, or a combination thereof.

12. The method of claim 11, wherein the at least one secondary cell is effective to promote localized vascularization in the patient.

13. The method of claim 1, wherein the cell-based composition is provided in a form selected from the group consisting of a matrix form and a capsule form.

14. The method of claim 13, wherein the form is effective to prevent degradation of the cell-based composition by an immunogenic cell for a period of time.

15. A cell-based therapy method, the method comprising the step of:
administering a cell-based composition to a patient with a plasma protein deficiency disorder to treat the plasma protein deficiency disorder, the cell-based composition comprising a mammalian adipose stromal cell capable of effectuating the promotion of a plasma protein within the patient;
wherein the mammalian adipose stromal cell comprises an isolated nucleotide sequence encoding a protein capable of compensating for the plasma protein deficiency disorder in the patient.

16. The method of claim 15, further comprising the step of differentiating the mammalian adipose stromal cell, wherein the differentiated mammalian stromal cell expresses at least one hepatocyte characteristic.

17. The method of claim 15, wherein the nucleotide sequence encodes a protein selected from the group consisting of Factor VIII, B-domainless Factor VIII, Factor VII, Factor IX, Factor X, protein C, and prothrombin.

18. The method of claim 15, wherein the mammalian adipose stromal cell is originally isolated from the patient that the differentiated adipose stromal cell is administered.

19. The method of claim 15, wherein the step of administering the mammalian adipose stromal cell is performed by a route selected from a group consisting of intravenous injection, intramuscular injection, subcutaneous injection, retrograde venous injection, arterial injection, surgical implantation, and intraocular placement.

20. The method of claim 15, wherein the mammalian adipose stromal cell is selected from the group consisting of a CD10+ mammalian adipose stromal cell, a CD13+ mammalian adipose stromal cell, a CD34+ mammalian adipose stromal cell, a CD34− mammalian adipose stromal cell, a CD45+ mammalian adipose stromal cell, a CD45− mammalian adipose stromal cell, a CD90+ mammalian adipose stromal cell, a CD90− mammalian adipose stromal cell, a CD140a+ mammalian adipose stromal cell, a CD140a− mammalian adipose stromal cell, a CD140b+ mammalian adipose stromal cell, and a CD140b− mammalian adipose stromal cell.

21. The method of claim 15, wherein the step of administering a cell-based composition comprises administering the cell-based composition comprising a mammalian adipose stromal cell, and at least one secondary cell selected from the group consisting of a mammalian endothelial cell, a mammalian endothelial progenitor cell, an unmodified adipose stromal cell, or a combination thereof.

22. The method of claim 21, wherein the at least one secondary cell is effective to promote localized vascularization in the patient.

23. The method of claim 15, wherein the cell-based composition is provided in a form selected from the group consisting of a matrix form and a capsule form.

24. The method of claim 23, wherein the form is effective to prevent degradation of the cell-based composition by an immunogenic cell for a period of time.

25. A composition to treat a plasma protein deficiency disorder, the composition comprising:
a mammalian adipose stromal cell;
wherein the composition is effective to treat the plasma protein deficiency disorder by effectuating the production of a plasma protein within the patient, the plasma protein being selected from the group consisting of Factor VIII, B-domainless Factor VIII, Factor VII, Factor IX, Factor X, protein C, and prothrombin.

26. The composition of claim 25, wherein the mammalian adipose stromal cell comprises an isolated nucleotide sequence encoding a protein capable of compensating for the plasma protein deficiency disorder in the patient.

27. The composition of claim 26, wherein the isolated nucleotide sequence encodes a protein selected from the group consisting of Factor VIII, B-domainless Factor VIII, Factor VII, Factor IX, Factor X, protein C, and prothrombin.

28. The composition of claim 25, wherein the composition is provided in a form selected from the group consisting of an intravenous injectable form, a surgically-implantable form, a intramuscular injectable form, a subcutaneous injectable form, a retrograde venous injectable form, an arterial injectable form, and an intraocular placeable form.

29. The composition of claim 25, further comprising a biologically-compatible carrier.

30. The composition of claim 25, wherein the mammalian adipose stromal cell is selected from the group consisting of a CD10+ mammalian adipose stromal cell, a CD13+ mammalian adipose stromal cell, a CD34+ mammalian adipose stromal cell, a CD34− mammalian adipose stromal cell, a CD45+ mammalian adipose stromal cell, a CD45− mammalian adipose stromal cell, a CD90+ mammalian adipose stromal cell, a CD90− mammalian adipose stromal cell, a CD140a+ mammalian adipose stromal cell, a CD140a− mammalian adipose stromal cell, a CD140b+ mammalian adipose stromal cell, and a CD140b− mammalian adipose stromal cell.

31. The composition of claim 25, further comprising:
at least one secondary cell selected from the group consisting of a mammalian endothelial cell, a mammalian endothelial progenitor cell, an unmodified adipose stromal cell, or a combination thereof.

32. The composition of claim 25, wherein the at least one secondary cell is effective to promote localized vascularization in the patient.

33. The composition of claim 25, wherein the composition is provided in a form selected from the group consisting of a matrix form and a capsule form.

34. The composition of claim 33, wherein the form is effective to prevent degradation of the cell-based composition by an immunogenic cell for a period of time.
